(19) United States
(12) Patent Application Publication
(10) Pub. No.: US 2020/0179341 A1
(43) Pub. Date: Jun. 11, 2020

(54) METHODS AND COMPOSITIONS FOR TREATING MULTIPLE MYELOMA AND INCREASING ANTIBODY DEPENDENT CELL CYTOTOXICITY BY TARGETING THE ARYL HYDROCARBON RECEPTOR

(71) Applicant: OHIO STATE INNOVATION FOUNDATION, Columbus, OH (US)

(72) Inventors: Don BENSON, Lewis Center, OH (US); Tiffany HUGHES, Columbus, OH (US); Robert J. LEE, Lewis Center, OH (US)

(73) Assignee: Ohio State Innovation Foundation, Columbus, OH (US)

(21) Appl. No.: 16/347,524

(22) PCT Filed: Nov. 6, 2017
(86) PCT No.: PCT/US2017/060201

§ 371 Date: May 3, 2019

Related U.S. Application Data
(60) Provisional application No. 62/417,510, filed on Nov. 4, 2016.

(51) Int. Cl. A61K 31/415; A61K 9/00; A61K 9/127; A61P 35/00

(52) U.S. Cl. CPC A61K 31/415; A61K 9/0019; A61K 45/06; A61P 35/00; A61K 9/1271

(57) ABSTRACT

Disclosed herein are methods and compositions for treating multiple myeloma, and for enhancing antibody-dependent cellular toxicity (ADCC). Specifically, disclosed herein are methods of using antagonists of the aryl hydrocarbon receptor (AHR) to treat multiple myeloma and enhance ADCC. Also specifically disclosed is liposomal CH233191.

### CROSS-REFERENCE TO RELATED APPLICATIONS

This application claims benefit of U.S. Provisional Application No. 62/417,510, filed Nov. 4, 2016, which is hereby incorporated by reference in its entirety.

### BACKGROUND

Over 20,000 people in the United States alone are diagnosed annually with multiple myeloma MM induces immune suppression, painful lytic bone disease and death most often related to infection or kidney failure. Novel therapies (such as the immunomodulatory agents lenalidomide and pomalidomide and agents targeting the proteosome including bortezomib and carfilzomib) have improved survival, but MM remains essentially incurable and is increasing in incidence.

Natural killer (NK) cells may play a key role in the immune response to MM; however, this effect is attenuated through specific MM immunoevasive strategies. However, despite advances in the understanding of the pathology of MM, there is still a need for compositions and methods for treating this disease. These needs and other needs are satisfied by the disclosed embodiments.

Monoclonal antibody developed to the specific cancer cell surface target can kill the cell with or without toxin attached just by binding to cell surface target. The antibody can initiate lysis of the cancer cell through apoptosis, complement dependent cytotoxicity (CDC) and antibody-dependent cellular cytotoxicity (ADCC). Monoclonal antibody therapy can be used to destroy malignant tumor cells and prevent tumor growth by blocking specific cell receptors or by delivering a conjugated toxin. Monoclonal antibody therapy is also useful in treating autoimmune disease, and graft vs. host disease. What is needed in the art are methods of enhancing ADCC in a subject in need thereof.

### SUMMARY

Disclosed herein is a composition comprising CH233191 and a liposome.

Also disclosed herein is a method of treating a plasma cell neoplasm, comprising: diagnosing a subject with a plasma cell neoplasm; and administering to the subject a substance that down-regulates aryl hydrocarbon receptor (AHR), thereby treating a plasma cell neoplasm.

Further disclosed is a method of diagnosing multiple myeloma in a subject, the method comprising detecting AHR levels in the subject.

Disclosed herein is a kit for treating a subject with multiple myeloma, the kit comprising CH233191 and an additional therapeutic agent for treating multiple myeloma.

Disclosed herein are methods for enhancing antibody dependent cellular cytotoxicity (ADCC) in a subject in need thereof.

### BRIEF DESCRIPTION OF THE FIGURES

The accompanying figures, which are incorporated in and constitute a part of this specification, illustrate several embodiments.

FIG. 1 A-C shows functional AHR is expressed in NK cell precursors

FIG. 2 shows that AHR antagonism promotes NK cell development and acquisition of cytokine production and cytotoxicity.

FIG. 3 shows AHR is expressed in MM cell lines, healthy plasma cells, and preferentially in primary MM cells from patient bone marrow. AHR antagonism abrogates AHR transcriptional function.

FIG. 4 shows the AHR gene expression is associated with decreased survival in MM.

FIG. 5 shows the AHR antagonism leads to MM cell death evident as early as 48-72 hours. Viability assessed after a 14 day exposure showed that cells exposed for 7 days were unable to recover from AHR antagonism after washout of the drug.

FIG. 6 shows AHR antagonism leads to selective, in vitro primary MM cell death. Representative results from n=2 patients with MM show effectiveness of AHR antagonism even in “high risk” MM. AHR antagonism also preferentially suppresses transcriptional activity of AHR (right panels) in primary MM cells.

FIG. 7 show AHR antagonism suppresses IL-6 and IL-6R in MM.

FIG. 8 shows AHR antagonism sensitizes MM cells to NK cell mediated lysis and enhances expression of NK cell activating ligands on MM cells.

FIG. 9 shows AHR antagonism with CH233191 appears to enhance Elotuzumab-mediated ADCC.

FIG. 10 shows AHR antagonism with CH233191 appears to enhance Daratumumab-mediated ADCC.

Additional advantages will be set forth in part in the description which follows, and in part will be obvious from the description. The advantages will be realized and attained by means of the elements and combinations particularly pointed out in the appended claims. It is to be understood that both the foregoing general description and the following detailed description are exemplary and explanatory only and are not restrictive, as claimed.

### DETAILED DESCRIPTION

The disclosed embodiments can be understood more readily by reference to the following detailed description and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in practice or testing, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the disclosed embodiments are not entitled to antedate such publication by virtue of prior invention.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, “noncancerous cells” can refer to cells that are normal or cells that do not exhibit any metabolic or physiological characteristics associated with cancer. For example, noncancerous cells are healthy and normal cells.

As used herein, the term “subject” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a patient. A patient refers to a subject afflicted with a disease or disorder, such as, for example, cancer and/or aberrant cell growth. The term “patient” includes human and veterinary subjects. In an aspect, the subject has been diagnosed with a need for treatment for cancer and/or aberrant cell growth.

The terms “treating”, “treatment”, “therapy”, and “therapeutic treatment” as used herein refer to curative therapy, prophylactic therapy, or preventative therapy. As used herein, the terms refer to the medical management of a subject or a patient with the intent to cure, ameliorate, stabilize, or prevent a disease, pathological condition, or disorder, such as, for example, cancer or a tumor. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In an aspect, the disease, pathological condition, or disorder is cancer, such as, for example, breast cancer, lung cancer, colorectal, liver cancer, or pancreatic cancer. In an aspect, cancer can be any cancer known to the art.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. For example, in an aspect, preventing can refer to the preventing of replication of cancer cells or the preventing of metastasis of cancer cells.

As used herein, the term “diagnosed” means having been subjected to a physical examination by a person of skill, for example, a physician or a researcher, and found to have a condition that can be diagnosed or treated by compositions or methods disclosed herein. For example, “diagnosed with cancer” means having been subjected to a physical examination by a person of skill, for example, a physician or a researcher, and found to have a condition that can be diagnosed or treated by a compound or composition that alleviates or ameliorates cancer and/or aberrant cell growth.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to cancer and/or aberrant cell growth) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a peptide, or a composition, or pharmaceutical preparation to a subject. Such methods are well known to those skilled in the art and include, but are not limited to, intracardiac administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed composition or peptide or pharmaceutical preparation and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the term “level” refers to the amount of a target molecule in a sample, e.g., a sample from a subject. The amount of the molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compare to a control or a sham or an untreated sample).

As used herein, the terms “effective amount” and “amount effective” refer to an amount that is sufficient to achieve the desired result or to have an effect on an undesired condition. For example, in an aspect, an effective amount of a peptide is an amount that kills and/or inhibits the growth of cells without causing extraneous damage to surrounding non-cancerous cells. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

By “modulate” is meant to alter, by increase or decrease. As used herein, a “modulator” can mean a composition that can either increase or decrease the expression level or activity level of a gene or gene product such as a peptide. Modulation in expression or activity does not have to be complete. For example, expression or activity can be modulated by about 10%, 20%, 30%, 40%, 50%, 60%, 70%, 80%, 90%, 95%, 99%, 100% or any percentage in between as compared to a control cell wherein the expression or activity of a gene or gene product has not been modulated by a composition.

An “AHR antagonist” refers to an AHR inhibitor that does not provoke a biological response itself upon specifically binding to the AHR polypeptide or polynucleotide encoding the AHR, but blocks or dampens agonist-mediated or ligand-mediated responses, i.e., an AHR antagonist can bind but does not activate the AHR polypeptide or polynucleotide encoding the AHR, and the binding disrupts the interaction, displaces an AHR agonist, and/or inhibits the function of an AHR agonist. Thus, as used herein, an AHR antagonist does not function as an inducer of AHR activity when bound to the AHR, i.e., they function as pure AHR inhibitors.

As used herein, “EC₅₀,” is intended to refer to the concentration or dose of a substance that is required for 50% enhancement or activation of a biological process, or component of a process, including a protein, subunit, organelle, ribonucleoprotein, etc. EC₅₀ also refers to the concentration or dose of a substance that is required for 50% enhancement or activation in vivo, as further defined elsewhere herein. Alternatively, EC₅₀ can refer to the concentration or dose of compound that provokes a response halfway between the baseline and maximum response. The response can be measured in an in vitro or in vivo system as is convenient and appropriate for the biological response of interest. For example, the response can be measured in vitro using cultured cancer cells or in an ex vivo organ culture system with isolated cancer cells.

Alternatively, the response can be measured in vivo using an appropriate research model such as rodent, including mice and rats. The mouse or rat can be an inbred strain with phenotypic characteristics of interest such as, for example, cancer and/or aberrant cell growth. As appropriate, the response can be measured in a transgenic or knockout mouse or rat wherein a gene or genes has been introduced or knocked-out, as appropriate, to replicate a disease process.

As used herein, “IC₅₀,” is intended to refer to the concentration or dose of a substance that is required for 50% inhibition or diminution of a biological process, or component of a process, including a protein, subunit, organelle, ribonucleoprotein, etc. IC₅₀ also refers to the concentration or dose of a substance that is required for 50% inhibition or diminution in vivo, as further defined elsewhere herein. Alternatively, IC₅₀ also refers to the half maximal (50%) inhibitory concentration (IC) or inhibitory dose of a substance. The response can be measured in an in vitro or in vivo system as is convenient and appropriate for the biological response of interest. For example, the response can be measured in vitro using cultured cancer cells or in an ex vivo organ culture system with isolated cancer cells. Alternatively, the response can be measured in vivo using an appropriate research model such as rodent, including mice and rats. The mouse or rat can be an inbred strain with phenotypic characteristics of interest such as, for example, cancer and/or aberrant cell growth. As appropriate, the response can be measured in a transgenic or knockout mouse or rat wherein a gene or genes has been introduced or knocked-out, as appropriate, to replicate a disease process.

The term “pharmaceutically acceptable” describes a material that is not biologically or otherwise undesirable, i.e., without causing an unacceptable level of undesirable biological effects or interacting in a deleterious manner. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “anti-cancer” or “anti-neoplastic” drug refers to one or more drugs that can be used in conjunction with an AHR antagonist or a composition comprising an AHR antagonist to treat cancer and/or aberrant cell growth.

The term “agent” as used herein in reference to an AHR antagonist means any compound or substance such as, but not limited to, a small molecule, nucleic acid, polypeptide, peptide, drug, ion, etc. An “agent” can be any chemical, entity, or moiety, including, without limitation, synthetic and naturally-occurring proteinaceous and non-proteinaceous entities. In some embodiments, an agent is a nucleic acid, a nucleic acid analogue, a protein, an antibody, a peptide, an aptamer, an oligomer of nucleic acids, an amino acid, or a carbohydrate, and includes, without limitation, proteins, oligonucleotides, ribozymes, DNAzymes, glycoproteins, siRNAs, lipoproteins, aptamers, and modifications and combinations thereof etc. In certain embodiments, as described herein, agents are small molecules having a chemical moiety. Compounds can be known to have a desired activity and/or property, e.g., modulate AHR activity, or can be selected from a library of diverse compounds, using, for example, the screening methods described herein.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g. , including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “enhance” as used herein means to improve the quality, amount, or strength of a phenomenon, especially a biological response.

The term “ADCC” or “antibody-dependent cell mediated cytotoxicity” is known in the art, and, as used herein, generally refers to a form of lymphocyte mediated cytotoxicity that functions only if antibodies are bound to the target cell. Antibody-coated target cells are killed by cells bearing Fc receptors specific for the Fc regions of the antibodies, especially NK cells.

The term “immuno-depleting agent” generally refers to a compound which results in a decrease in the number of cells of the immune system (such as lymphocyte) when administrated to an individual. Examples include, but are not limited to, chemotherapeutic agents.

The term “immuno-therapeutic agent” generally refers to a compound which results in the activation of an immune response when administrated to an individual. Examples include, but are not limited to, tumor antigens or monoclonal therapeutic antibodies.

### Methods and Compositions

Disclosed are the components to be used to prepare a composition of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

All patents, patent applications, and other scientific or technical writings referred to anywhere herein are incorporated by reference in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed can be resorted to by those skilled in the art, and that such modifications and variations are considered to be within the scope of this invention as defined by the description and the appended claims.

General

Neoplasms are abnormal cell growths, which may be cancerous or benign. Plasma cells (also referred to as plasma B cells or plasmocytes) develop from mature B lymphocytes (B cells), and are normally involved in secreting antibodies in order to fight foreign elements in the body (e.g., bacteria or virus infections). The presence of plasma cell neoplasms can result in less active healthy red blood cells, white blood cells, and platelets. This condition may cause anemia or easy bleeding, or make it easier to get an infection. The abnormal plasma cells often form tumors in bones or soft tissues of the body. The plasma cell neoplasms may also produce a large amount of a single antibody, called M protein (or monoclonal protein, myeloma protein or paraprotein), that is not needed by the body, does not help fight infection and can cause damage to the kidneys. In some cases, malignant plasma cells lose the ability to make and match heavy chains and light chains so that kappa and lambda light chains (also called Bence Jones protein) leave the cell unattached into the blood and are excreted in the urine. Examples of plasma cell neoplasms include multiple myeloma (MM), solitary plasmacytoma of the bone (SPB), plasma cell leukemias, AL amyloidosis, and extramedullary plasmacytomas (EMP).

Multiple myeloma (MM) is a malignancy characterized by the expansion of plasma B cells that produce monoclonal immunoglobulin (e.g., IgG, IgA, IgD, IgE, or free kappa or lambda light chains). The overall survival of patients with MM varies greatly from a few months to many years; the mean is approximately five years. Anemia, hypercalcemia and bone lesions correlate directly with total mass of myeloma cells and have important prognostic significance. Other prognostic factors include age, the plasma cell labeling index, serum beta2 -microglobulin, C-reactive protein, thymidine kinase, and soluble interleukin-6 receptor. Major complications, such as infection and renal insufficiency, are the most common causes of death for myeloma patients. Almost all patients with multiple myelomas have a risk of eventual relapse (Kyle, R K et al., (2004) N Engl J Med 351: 1860-1873).

It has been shown that the aryl hydrocarbon receptor (AHR), a ligand-binding transcription factor, mediates NK cell development, and AHR can be involved in MM pathobiology. Antagonism of AHR in innate lymphoid precursors enhances NK cell development and acquisition of cytolytic properties and, at the same time, appears to impair MM cell viability. AHR can therefore be a target for therapeutic development in MM.

The AHR is a transcription factor originally identified by its sensitivity to polycyclic aromatic hydrocarbons which activate its function in regulating xenobiotic-metabolizing enzymes (Denison Chem Biol Interact 2002). In the healthy setting, inactive AHR in the cytoplasm translocates to the nucleus upon endogenous ligand binding to dimerize with the AHR nuclear translocator (ARNT), promoting the transcription of genes involved in lymphopoiesis (Kadow J I 2011; Veldholen Nature 2008). AHR is expressed to varying degrees in B-cell development from CD34(+) stem cells through terminally differentiated plasma cells, which show the greatest relative expression (Sherr Semin Immunopathol 2013). AHR can also play roles in T-cell differentiation, modulating regulatory T cell (T_{reg}) development and Th17 polarization (Quintana Nature 2008). AHR therefore has an integral role in natural killer (NK) cell development (Hughes Cell Reports 2014). AHR has been implicated in a number of solid tumors (particularly in aggressive, advanced stages (Opitz Nature 2011; Richmond PLoS One 2014; Safe Toxicol Sci 2013).

AHR directly transcribes Th17 cytokines, which are elevated in MM (Prabhala Blood 2010) as well as other cytokines intimately important in MM biology, including: interleukin (IL)-1β, IL-21, TGF-β, and IL-6 (DiNatale J Bio Chem 2010; Lahoti J Pharmacol Exp Ther 2014; Gramatzki Oncogene 2009; Apetoh Nat Immunol 2010). In fact, IL-6 transcription is synergistically enhanced via AHR in the presence of a second inflammatory stimulus in multiple solid tumor models (DiNatale J Bio Chem 2010; Hollingshead Cancer Res 2008). Also, in addition to collaborating with other transcription factors directing normal plasma cell differentiation (Sherr Semin Immunopathol 2013), AHR also cooperates with at least three transcription factors critical to MM biology. AHR interacts with c-maf (Apetoh Nat Immunol 2010), a transcription factor implicated in the t(14;16) mutation conferring “high risk” cytogenetic designation in MM. Even in the absence of t(14;16), c-maf is expressed in many cases of MM, which promotes MM/stromal cell interaction via integrin β7, a process directly facilitated by AHR/c-maf cooperation (Hurt Cancer Cell 2004; Monteiro Biochem Biophys Res Commun 2007).

In addition, there appears to be a reciprocal, facilitative relationship between AHR and NF-κB family members, also important in MM (Chesi Int J Hematol 2013). AHR expression is enhanced by the NF-κB heterodimer, RelA-p50, for which a specific functional response element is present in the proximal promoter of AHR (Vogel J Biol Chem 2014). In turn, AHR interacts with NF-κB in the cellular response to polycyclic hydrocarbons (Tian J Biol Chem 1999) and partners with NF-κB member, RelB (important in MM survival) (Cormier PLoS One 2013), in the production of chemokines and cytokines in cancer (Vogel Arch Biochem Biophys 2011; Vogel Biochem Biophys Res Commun 2007).

AHR, through functional cooperation with the RelA NF-βB subunit, also induces c-myc overexpression in cancer (Kim Oncogene 2000). That AHR and NF-κB cooperation is associated with c-myc is a compelling indictment of AHR in MM pathogenesis and progression as increased c-myc expression is common in newly diagnosed MM and may be directly responsible for progression from monoclonal gammopathy of undetermined significance (MGUS) to MM (Chesi Int J Hematol 2013). Most advanced cases of MM are associated with c-myc abnormalities (Chesi Int J Hematol 2013), and c-myc abnormalities also appear to signify a “high risk” clinical course (Gilitza Leuk Lymphoma 2014). In addition, it has been demonstrated that p53 expression is modulated by microRNAs in MM (Pichiorri Cancer Cell 2010).

AHR has been implicated in microRNA biology as well, showing another role for AHR in MM via modulation of microRNA expression important in the development and progression of MM (Gordon Mol Carcinog 2014). Finally, the endogenous AHR agonists kynurenine (KYN) and kynurenic acid (KYNA) are both elevated in patients with MM (Mariani Bone Abstracts 2013; Bonanno J Transl Med 2012; Zdzisinska Leuk Res 2010), and can be produced directly by MM cells, showing an autocrine feedback for AHR function. In addition, KYNA levels in patients with MM correlate with ISS stage and advanced disease (Zdzisinska Leuk Res 2010).

Therefore, AHR is integral to MM development and pathology, including underlying myelomagenesis as well as progression from the asymptomatic MGUS precursor state to advanced disease stages and “high risk” subtypes (which represent a significant area of unmet, therapeutic medical need). AHR antagonism causes MM cell lysis and shows efficacy even in primary MM samples obtained from patients with high risk disease (Example 1). Furthermore, AHR antagonism can yield favorable immunomodulatory effects on immune cell subsets and the microenvironment cytokine milieu: stimulating hematopoiesis (Boitano Science 2010), promoting NK cell development and cytotoxicity (Hughes Cell Reports 2014), and counteracting factors involved in MM-induced immune dysregulation (Negishi J Immunol 2005). Third, AHR antagonism can augment the function of tumor-directed, therapeutic monoclonal antibodies that exert efficacy via antibody-dependent cellular cytotoxicity (ADCC) through the parallel, facilitative effects on MM and immune cells. The liposomal formulation of CH233191, for example, is innovative in circumventing challenges with drug delivery and overcoming prior barriers to development.

Methods of Treatment

Disclosed herein are methods of treating a plasma cell neoplasm, comprising diagnosing a subject with a plasma cell neoplasm; and administering to the subject a substance that down-regulates aryl hydrocarbon receptor (AHR), thereby treating a plasma cell neoplasm. As disclosed above, the plasma cell neoplasm can be multiple myeloma (MM), solitary plasmacytoma of the bone (SPB), plasma cell leukemias, AL amyloidosis, and extramedullary plasmacytomas (EMP).

A subject can be diagnosed with MM by a variety of methods known to those of skill in the art (Rajikumar Lancet Oncology 2014). Similarly, progression or survival rates can be measured based on these criteria. For example, clonal bone marrow plasma cells can be shown to be greater than or equal to 10%, or biopsy-proven bone or extramedullary plasmacytoma and any one or more of the following myeloma defining events can be shown: evidence of end organ damage due to the underlying plasma cell neoplasm (hypercalcemia (>11 mg/dL) or 1 mg/dL greater than institutional normal range, anemia (hemoglobin<10 g/dL or >2 g/dL below normal value), renal insufficiency (CrCl<40 or serum creatinine>2 mg/dL), bone lesions (one or more osteolytic lesions on skeletal radiography, CT or PET/CT)). MM can also be diagnosed in cases where clonal bone marrow plasma cells are greater than or equal to 60%, or the involved/uninvolved serum free light chain ratio is greater than or equal to 100. It can also be diagnosed by the finding of more than one focal lesion on MRI.

AHR can be regulated by way of an antagonist (also referred to as an inhibitor or down-regulator herein). Methods of treating a subject with a plasma cell neoplasm comprising administering to the subject an effective amount of an AHR antagonist are also therefore disclosed. For example, one substance that can down-regulate AHR is CH233191. This drug can be in a liposomal formulation, for example. CH233191 is discussed in more detail below. Another example of a substance that antagonizes AHR is stemreginin-1. Therefore, disclosed herein are methods of treating a subject with MM, comprising administering CH233191 or stemreginin-1 to the subject.

Also disclosed is a method of treating a plasma cell neoplasm, comprising: diagnosing a subject with a plasma cell neoplasm; and administering to the subject a substance that regulates multiple myeloma stem cells. Cancer stem cells, as a field, are conceived of as having relatively low proliferative potential and the capacity for drug resistance, and this subset of cells has been indicted in the relapsing nature of cancer and the incurability of forms of the disease. Data has shown that MM stem cells can potentiate the disease. Therefore, disclosed is a method of reducing, or down-regulating, MM stem cells or their production, as MM stem cells can express AHR. Therefore, antagonizing AHR can regulate MM stem cells.

Also disclosed is a method of treating a plasma cell neoplasm, comprising: diagnosing a subject with a plasma cell neoplasm; and administering to the subject a substance that modulates regulatory T cell (T_{reg}) development, thereby treating a plasma cell neoplasm. T_{regs} are known to be potent suppressors of immunity. AHR antagonism can not only have a favorable direct effect on killing and/or suppressing MM cells, and a favorable indirect effect of enhancing NK cell development and/or cytotoxicity, but thirdly, can favorably modulate the T_{reg} population. For example, the T_{reg} population can be reduced by 10, 20, 30, 40, 50, 60, 70, 80, 90, or 100%.

Other AHR antagonists include the synthetic flavonoid, 3′-methoxy-4′nitroflavone (“3M4NF”), and the indole derivative 3,3′-diindolylmethane (“DIM”). These compounds have been shown to function through direct competition for binding to the AHR ligand binding site (Henry et al., Mol. Pharmacol. 55:716-725 (1999); Hestermann et al., Mol. Cell. Biol 23:7920-7925 (2003)). Also disclosed are CB7993113 (Parks et al. Mol Pharm 2014), CB9950998, and CMLD-2166, which modulate AHR activity by functioning as AHR antagonists. Also disclosed is GNF-351, which has been given via oral administration in an in vivo model (Fang Brit Pharmacol 2014). Disclosed are the use of these drugs in a liposome, for example.

Accordingly, an AHR modulator refers to an agent, such as a small molecule that modulates or causes or facilitates a qualitative or quantitative change, alteration, or modification in one or more processes, mechanisms, effects, responses, functions, activities or pathways mediated by the AHR receptor. Specifically, an AHR antagonist (or inhibitor, used interchangeably throughout) refers to an agent, such as a small molecule, that causes a decrease in, inhibition of, or diversion of, constitutive activity of the AHR.

An AHR antagonist can bind to the AHR. As used herein, “selectively binds” or “specifically binds” refers to the ability of an AHR antagonist, described herein to bind to a target, such as the AHR polypeptide, with a KD 10⁵ M (10000 nM) or less, e.g., 10⁶ M or less, 10⁷M or less, 10⁸ M or less, 10⁹ M or less, 10¹⁰ M or less, 10¹¹ M or less, or 10¹² M or less. For example, if an antagonist as described herein binds to the AHR polypeptide with a KD of 10⁵ M or lower, but not to other molecules, or a related homologue, then the agent is said to specifically bind the AHR polypeptide. Specific binding can be influenced by, for example, the affinity and avidity of the antagonist and the concentration of the antagonist used. The person of ordinary skill in the art can determine appropriate conditions under which the antagonists described herein selectively bind using any suitable methods, such as titration of an AHR antagonist in a suitable cell binding assay, such as those described herein.

With respect to the AHR target, the term “ligand interaction site” on the AHR means a site, epitope, antigenic determinant, part, domain or stretch of amino acid residues on the AHR that is a site for binding to a ligand, receptor or other binding partner, a catalytic site, a cleavage site, a site for allosteric interaction, a site involved in multimerisation (such as homomerization or heterodimerization) of the AHR; or any other site, epitope, antigenic determinant, part, domain or stretch of amino acid residues on the AHR that is involved in a biological action or mechanism of the target, i.e., the AHR. More generally, a “ligand interaction site” can be any site, epitope, antigenic determinant, part, domain or stretch of amino acid residues on the AHR polypeptide to which an antagonist described herein can bind, such that AHR activity and/or expression is (and/or any pathway, interaction, signalling, biological mechanism or biological effect in which the AHR is involved) is modulated.

The terms “inhibit,” “decrease,” and “reduce”, are all used herein generally to mean a decrease by a statistically significant amount. Accordingly, AHR down regulation is considered to be achieved when the activity value of an AHR polypeptide, or a polynucleotide encoding the AHR is about at least 10% less, at least 20% less, at least 30% less, at least 40% less, at least 50% less, at least 60% less, at least 70% less, at least 80% less, at least 90% less, at least 95% less, at least 98% less, at least 99% less, up to including 100% or less, i.e., absent, or undetectable, in comparison to a reference or control level in the absence of the inhibitor. In some embodiments of the aspects described herein, the AHR inhibitors inhibit constitutive AHR activity.

In addition to an AHR antagonist, also disclosed is a method of administering to the subject one or more additional therapeutic agents for treating plasma cell neoplasms, or for enhancing ADCC. The additional therapeutic agent or ADCC treatment can administered simultaneously with the AHR inhibitor, after the AHR inhibitor, or before the AHR inhibitor. For example, the additional therapeutic agent can be administered months, weeks, days, hours or minutes before the AHR inhibitor, or months, weeks days, hours, or minutes after the AHR inhibitor. It can be administered multiple times throughout a course of administration, such as before, during, and after administration with an AHR inhibitor.

Specifically, disclosed are antibodies that can have combinatorial efficacy and/or augmentation of the monoclonal antibodies' direct anti-MM effect with liposomal CH233191 or another AHR antagonist. Examples of anti-cancer drugs or anti-neoplastic drugs that can be used to treat multiple myeloma include, but are not limited to, the following: elotuzumab, daratumumab, silotuximab, isatuximab, rituximab, and milatuzumab. Other antibodies in development for myeloma include, but are not limited to: lirilumab, urelumab, ulocuplumab, nivolumab, pembrolizumab, indatuximab, lucatumumab, dacetuzumab, durvalumab, and IPH2201.

The additional therapeutic agent can also comprise any known agent for treating plasma cell neoplasms. Liposomal CH233191, as well as other AHR antagonists, can be used in combination with any other therapy for MM. Examples include, but are not limited to, immunomodulating agents (thalidomide, lenalidomide, pomalidomide), proteosome inhibitors (bortezomib, carfilzomib and ixazomib), as well as the epigenetic agent panobinostat. Cytotoxic chemotherapies for MM include, but are not limited to, melphalan, cyclophosphamide, carmustine, and liposomal doxorubicin. The corticosteroids dexamethasone and prednisone can be used in conjunction with the agents disclosed herein. Also disclosed is the use of bisphosphonates, such as pamidronate and zolendronic acid.

Also disclosed herein are methods of enhancing antibody-dependent cell-mediated cytotoxicity (ADCC), also referred to herein as antibody-dependent cellular cytotoxicity. ADCC is a mechanism of cell-mediated immune defense whereby an effector cell of the immune system actively lyses a target cell, whose membrane-surface antigens have been bound by specific antibodies. ADCC requires an effector cell which classically is known to be natural killer (NK) cells that typically interact with IgG antibodies. However, macrophages, neutrophils and eosinophils can also mediate ADCC.

The method for enhancing ADCC disclosed herein permits the treatment of cancers, auto-immune diseases, tissue graft or organ rejections, including graft versus host disease, and infectious diseases. Indeed, ADCC plays a major role in such diseases or conditions for the elimination of infected cells as well as tumor cells.

Said immunotherapeutic agent can also comprise monoclonal therapeutic antibodies. Examples of monoclonal antibodies include, but are not limited to, Infliximab (anti-TNFα), Basiliximab, Daratumumab, Elotuzumab, Milatuximab, Silotuximab, Isatuximab, Daclizumab (anti-CD25), Trastuzumab (anti-Her2/neu), Rituximab, Ibritumomab tiutexan (anti-CD20), Tositumomab (anti-CD122), Gemtuzumab ozogamicin (anti-CD33), Alemtuzumab (anti-CD52). Such agents can be administrated before, during or after administration of CH233191.

Said method for enhancing ADCC can further comprise the administration of at least one immuno-depleting agent. Said immuno-depleting agents can be a chemotherapeutic agent. Examples of chemotherapeutic agents include, but are not limited to, 5-fluorouracil, aziathioprine, cyclophosphamide, anti-metabolites (such as fludarabine), anti-neoplastics (such as etoposide, doxorubicin, methotrexate, vincristine), prednisone, carboplatin, cis-platinum and the taxanes such as taxol.

The method for enhancing ADCC according to the invention can permit the treatment of cancer in combination with antitumoral vaccination. The method for enhancing ADCC can permit the treatment of cancer, especially solid tumors, in combination with monoclonal antibody therapy. Solid tumors, such as sarcomas and carcinomas, comprise fibrosarcoma, myxosarcoma, liposarcoma, chondrosarcoma, osteogenic sarcoma, colon carcinoma, lymphoid malignancy, pancreatic cancer, breast cancer, lung cancer, ovarian cancer, prostate cancer, renal cell carcinoma, melanoma, and CNS tumors can be treated using the methods disclosed herein.

Examples of monoclonal antibody used for treating solid tumors include but are not limited to Trastuzumab used for treating breast cancer or Rituximab, Ibritumomab tiutexan or Tositumomab for treating lymphoma.

Disclosed is a method for enhancing ADCC for the treatment of cancer, especially haematological tumors, optionally in combination with monoclonal antibody therapy. The method comprises the administration of CH233191 in combination with at least one monoclonal antibody used for treating hematologic or lymphoid malignancies.

Hematological tumors comprise acute lymphocytic leukaemia, acute myelogenous leukaemia, chronic lymphocytic leukaemia, chronic myelogenous leukaemia, indolent non Hodgkin's lymphoma, high-grade Hodgkin's lymphoma, Hodgkin's lymphoma, multiple myeloma or myelodysplastic syndrome. Examples of monoclonal antibody used for treating hematologic or lymphoid malignancies include, but are not limited to, Gemtuzumab ozogamicin used for treating acute myelogenous leukaemia, or Alemtuzumab used for treating chronic lymphocytic leukaemia.

Disclosed is a method for enhancing ADCC for the treatment of autoimmune diseases, optionally in combination with monoclonal antibody therapy. Said method comprises the administration of CH233191 with at least one monoclonal antibody used for treating autoimmune diseases. Autoimmune diseases comprise type I diabetes, multiple sclerosis, systemic lupus erythemateous, thyroiditis, rheumatoid arthritis. Examples of monoclonal antibody used for treating autoimmune diseases include but are not limited to Infliximab used for treating polyarthrite rhumatoïde or Cröhn disease.

Also disclosed is a method for enhancing ADCC for the treatment of tissue graft or organ rejection, including graft versus host disease (GVHD), optionally in combination with monoclonal antibody therapy. Said method comprises the administration in an individual in need thereof CH233191 in combination with at least one monoclonal antibody used for treating tissue graft or organ rejection.

Grafts, referring to biological material derived from a donor for transplantation into a recipient, include such diverse material as, for example, isolated cells such as islet cells and neural-derived cells, tissue such as the amniotic membrane of a newborn, bone marrow, hematopoietic precursor cells, and organs such as skin, heart, liver, spleen, pancreas, thyroid lobe, lung, kidney, or tubular organs. Examples of monoclonal antibody used for treating tissue graft or organ rejection include but are not limited to Basiliximab or Daclizumab used for treating kidney rejection.

Further disclosed is a method for enhancing ADCC for the treatment of infectious diseases, especially bacterial and viral infections.

In an aspect, a disclosed AHR antagonist, such as CH233191, can be administered to a subject repeatedly. In an aspect, a disclosed composition can be administered to a subject at least two times. In an aspect, a disclosed composition can be administered to the subject two or more times. In an aspect, a disclosed composition can be administered at routine or regular intervals. For example, in an aspect, a disclosed composition can be administered to the subject one time per day, or two times per day, or three or more times per day. In an aspect, a disclosed composition can be administered to the subject daily, or one time per week, or two times per week, or three or more times per week, etc. In an aspect, a disclosed composition can be administered to the subject weekly, or every other week, or every third week, or every fourth week, etc. In an aspect, a disclosed composition can be administered to the subject monthly, or every other month, or every third month, or every fourth month, etc. In an aspect, the repeated administration of a disclosed composition occurs over a pre-determined or definite duration of time. In an aspect, the repeated administration of a disclosed composition occurs over an indefinite period of time.

Methods of Diagnosing

Also disclosed are methods of diagnosing multiple myeloma in a subject. For example, disclosed is a method of detecting AHR levels in the subject. Elevated AHR levels can be used as both a predictor of disease, or as a marker of progression of the disease. For example, elevated AHR levels can correlate with progression from asymptomatic precursor state (e.g., MGUS or smoldering myeloma) active disease. For example, an increase in AHR levels by 10, 20, 30, 40, 50, 60, 70, 80, 90, or 100% in an individual over a given period of time, or an increase by 1.5, 2, 3, 4, 5, 6, 7, 8, 9, 10, 11, 12, 13, 14, 15, 16, 17, 18, 19, or 20 fold or higher, can indicate that the subject is at risk of developing, or has, multiple myeloma. It can also mean that the subject has progressed from one stage of MM to another. For example, the subject can have SMM (smoldering MM) and detecting the level of AHR in the subject can indicate that the subject has progressed to another stage of development of MM. Detection of the level of AHR in a subject can also be used to determine the survival rate of a subject, such as a projection of life expectancy. It can also be used to determine a subject's response to therapy.

Detecting AHR levels can be done in conjunction with other measurements for diagnosing MM or determining stage/level/prognosis of a subject who has already been diagnosed with MM. Examples include those given above for determining plasma cell neoplasms in a subject, such as percentage of plasma cells in bone marrow, or a plasma cell tumor. Other tests can include, but are not limited to, urinalysis, computed tomography (CT) scan, or magnetic resonance imaging (MRI).

Kits

Kits for practicing the methods disclosed herein are further provided. By “kit” is intended any manufacture (e.g., a package or a container) comprising at least one compound or composition disclosed herein. The kit may be promoted, distributed, or sold as a unit for performing the methods of the present disclosure. Additionally, the kits may contain a package insert describing the kit and methods for its use. Any or all of the kit reagents may be provided within containers that protect them from the external environment, such as in sealed containers or pouches.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, e.g., about 1 to 100 mg/kg; intranasal instillation, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer drugs, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

Specifically disclosed herein are kits for treating a subject with multiple myeloma, the kit comprising CH233191 and an additional therapeutic agent for treating multiple myeloma. The kit can also comprise an additional therapeutic agent. Examples of such therapeutic agents are daratumumab, elotuzumab, silotuximab, isatuximab, rituximab, or milatuzumab.

Liposomal Compositions

Disclosed herein is a composition comprising CH233191. CH223191 has the following formula:

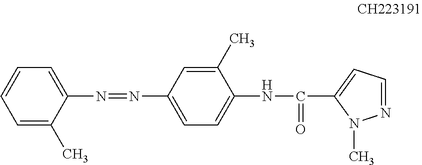 [SMILES: Cc1ccccc1N=Nc1ccc(NC(=O)c2ccnn2C)c(C)c1]

Disclosed herein is CH233191 and a liposome, referred to herein as “liposomal CH233191.” In a liposome-drug delivery system, a bioactive agent such as a drug is entrapped in the liposome and then administered to the subject to be treated. For example, Rahman et al., U.S. Pat. No. 3,993,754; Sears, U.S. Pat. No. 4,145,410; Paphadjopoulos et al., U.S. Pat. No. 4,235,871; Schneider, U.S. Pat. No. 4,224,179; Lenk et al., U.S. Pat. No. 4,522,803; and Fountain et al., U.S. Pat. No. 4,588,578. As disclosed herein, encapsulation of CH233191 improved the therapeutic benefits by facilitating its solubility, tumor localization, and preventing its degradation and excretion among others.

One specific advantage of liposomal CH233191 is that it can confer anti-MM benefit without the need for concomitant use of corticosteroids. Steroid-free regimens are a key unmet medical need, as these agents confer a great deal of toxicity and morbidity in an already vulnerable patient population, e.g., immune suppression, hypertension, glucose intolerance, osteoporosis, psychiatric effects, fluid retention, cataracts, etc.) Liposomal CH233191, either alone or in combination, can provide effective anti-MM benefit without the need for corticosteroids.

Liposomes are self-assembling phospholipid bilayer structures that can be prepared from phospholipids from natural or synthetic sources. These vesicles can encapsulate water soluble molecules in the aqueous volume while water insoluble molecules can be embedded in the hydrophobic region of the lipid bilayer. The simplest and the most widely used method for preparing liposomes is still the thin lipid film hydration method. The constituents of a liposomal delivery system are the primary determinants of the preparation method to be employed. For instance; hydrophobic molecules can be included during the lipid film formation process (passive loading), whereas water soluble molecules can be introduced during the hydration step (passive loading) or incorporated later on by active loading procedures using ion gradients. The phospholipid backbone of the liposomes consists of saturated or unsaturated phospholipids with acyl chain length of 14 to 20 carbons. Surface modification by hydrophilic polymers is a commonly used method in liposomal delivery systems. The main goals of surface modification are prevention of particle aggregation and reduction of the capture of the liposomes by cells of the reticuloendothelial system, due to steric hindrance of protein adsorption provided by the polymers.;

For intravenous delivery route, the size of colloidal drug delivery systems (DDS) should preferably be below 400 nm in order to prevent opsonization and thereby activation of the complement system and to facilitate extravasation at the site of the tumor. The CH233191 liposome formulation disclosed herein can be between 40 and 200 nm. Preferably, the formulation can be between 50 and 150 nm. Still more preferably, the formulation can be between 80 and 130 nm. Unlike low molecular weight small molecule drugs that tend to be cleared rapidly from blood circulation, drug carrier systems including liposomes have the crucial advantage of long circulation times enabling higher intratumor concentrations. The encapsulation efficiency of the liposomal CH233191 disclosed herein can be over 75%, 80%, or 90%.

Liposomal CH233191 can provide for controlled release of the drug. The liposomal formulation allows the half life of CH233191 release to be selectively varied, to provide release for a selected period of up to several days. CH233191 can then be given less often and without the sharp fluctuations seen when free drug injections are used. Further, a greater degree of control can be achieved with liposome formulations than which have been proposed heretofore.

Pharmaceutical Compositions

Disclosed herein is a pharmaceutical composition comprising an AHR antagonist, such as CH233191, in a pharmaceutically acceptable carrier. Specifically, disclosed is liposomal CH233191.

Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. P P. Gerbino, Lippincott Williams & Wilkins, Philadelphia, Pa. 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. A typical daily dosage of the disclosed composition used alone might range from about 1 μg/kg to up to 100 mg/kg of body weight or more per day, depending on the factors mentioned above.

In some embodiments, the molecule is administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of molecule administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the disclosed embodiments.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: AHR Antagonism and Multiple Myeloma

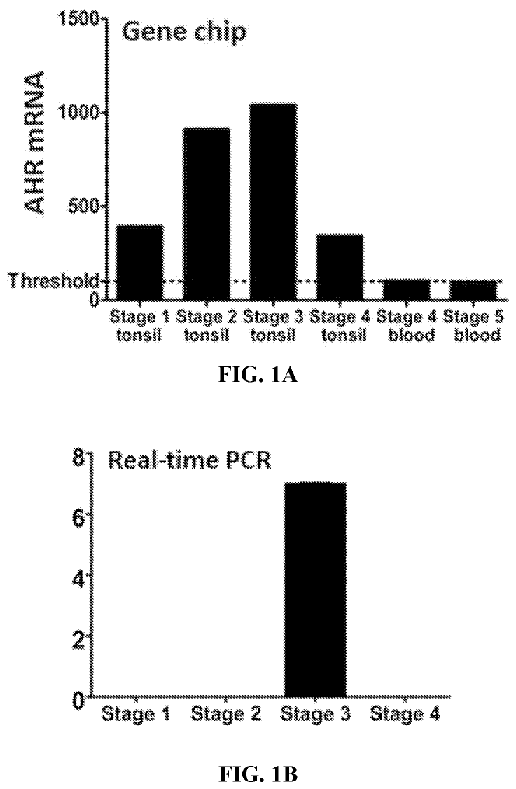

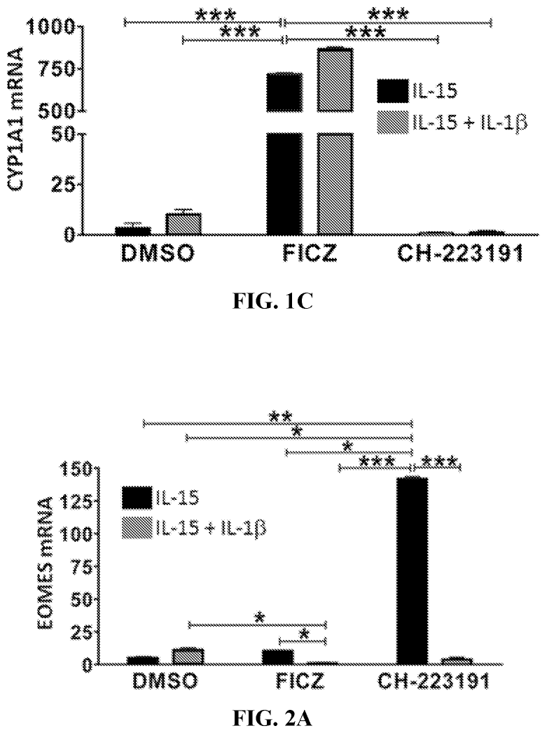

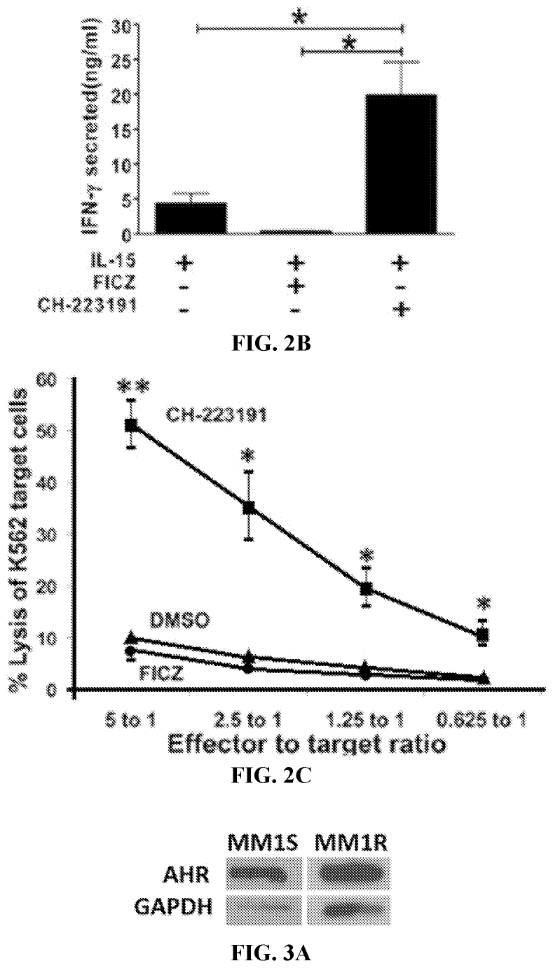

AHR antagonism promotes the development of functionally mature NK cells from a distinct innate lymphoid cell (ILC). The development of NK cells from an ILC stage that expresses functional AHR, indicated by CYP1A1 expression (FIG. 1) was previously described (Hughes Immunity 2010). In this precursor population, antagonism of AHR increased expression of EOMES (an NK-cell specific lineage transcription factor, FIG. 2) and promoted CD56 and CD94 expression, markers of NK cell maturity (Hughes Cell Reports 2014). As compared to control and AHR agonist (FICZ) treated cells, treatment with the AHR antagonist (CH-CH223191 led to rapid acquisition of cytokine production as well as cytotoxicity capabilities (FIG. 2). Results with another AHR antagonist, Stemreginin 1, as corroborated this finding (Roeven Blood 2014).

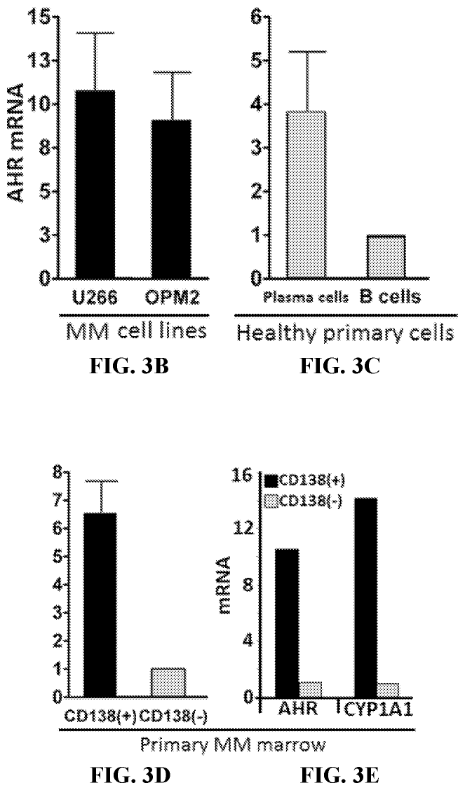

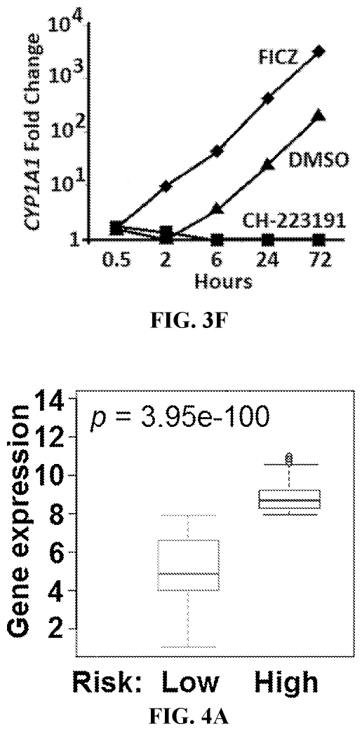

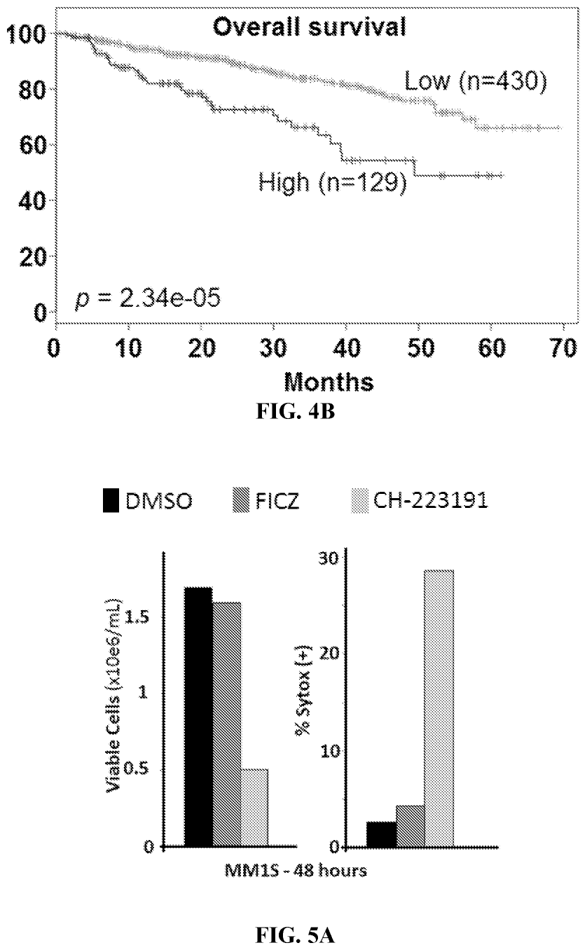

Functional AHR is expressed in MM cell lines and primary MM cells. AHR protein and transcript is expressed in MM cell lines, healthy donor plasma cells, and in primary MM cells (n=2 donors, FIG. 3). Importantly, AHR is functional and expression is greater in MM tumor cells compared to autologous CD138(−) cells (FIG. 3). AHR agonism increases the transcription of CYP1A1 whereas AHR antagonism suppresses CYP1A1 expression, demonstrating that AHR is functional in MM cells (FIG. 3, right panel). In addition, in analysis of a public gene expression database of samples from newly diagnosed MM patients, it was found that AHR expression correlates with survival (FIG. 4).

AHR drives expression of an immunophenotype associated with development of normal and malignant plasma cells. MM tumor cells recapitulate at least some aspects of normal plasma cell appearance and function (Shapiro-Shelef, Curr Opin Immunol 2004; Zhan Blood 2003). Treatment of MM cells with an AHR agonist leads to diminished expression of differentiation markers, including CD38, CD56, and CD138 and upregulation of CD10, CD11a, CD13, CD19, CD20, CD27, CD40, CD45 and CD117. This phenotype is reminiscent of highly proliferative, MM clonogeneic progenitor cells described by several groups, described as being more “stem-cell like” and more drug resistant (Kawano Int J Oncol 2012; Matsui Blood 2004; Pellat-Deceunynck Blood Cells Mol Dis 2004; Medina Blood 2002; Matwui Cancer Res 2008). As the CD45(+) plasma cell subset appears to have the greatest proliferative potential (Pellat-Deceunynck Blood Cells Mol Dis 2004), AHR antagonism can be a useful strategy to promote a more differentiated phenotype with less proliferative capacity and greater therapeutic susceptibility.

AHR antagonism can target multiple myeloma stem cells (MMSC) (also called “multiple myeloma propagating cells” (MMPC)). Cancer stem cells, as a field, are conceived of as having relatively low proliferative potential and the capacity for drug resistance, and this subset of cells has been indicted in the relapsing nature of cancer and the incurability of forms of the disease. Data has shown that MM stem cells can potentiate the disease. Interestingly, in myeloma, these “stem cells” or “propagating cells” are typically not described as CD138(+) plasma cells (like the vast majority of the tumor), but rather as B cells (CD19+, CD27+, ALDH1A1+). MM stem cells can express AHR, and therefore liposomal CH233191 and other AHR antagonists can be used as therapy to kill MM stem cells.

AHR expression can modulate other cell subsets in MM. Regulatory T cells (T_{regs}) can also express AHR in MM. T_{regs} are known to be potent suppressors of immunity. AHR antagonism with liposomal CH233191 can not only have a favorable direct effect on killing and/or suppressing MM cells, and a favorable indirect effect of enhancing NK cell development and/or cytotoxicity, but thirdly, can favorably modulate the Treg population.

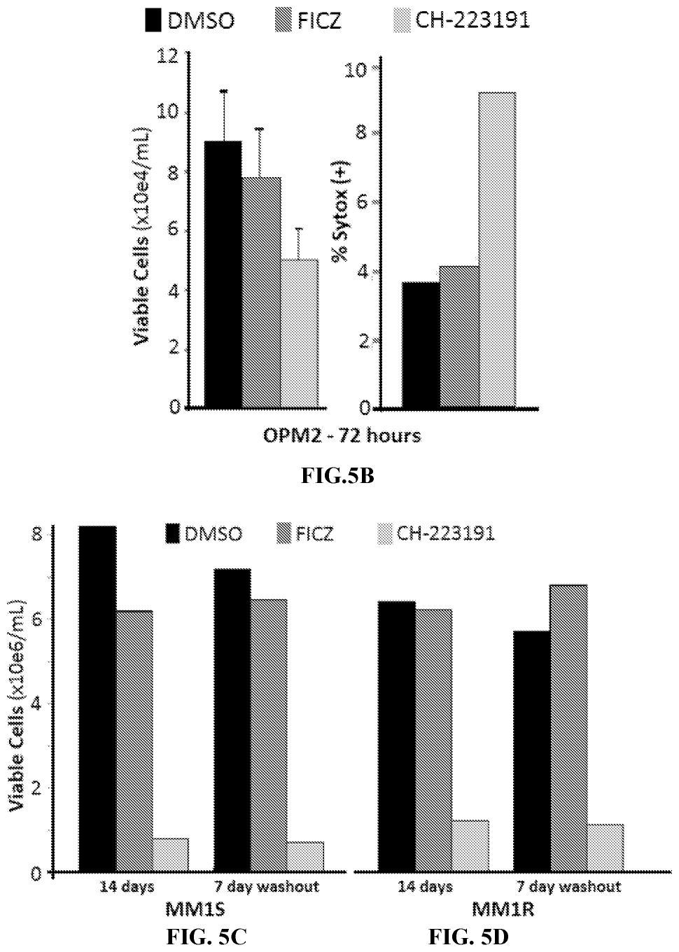

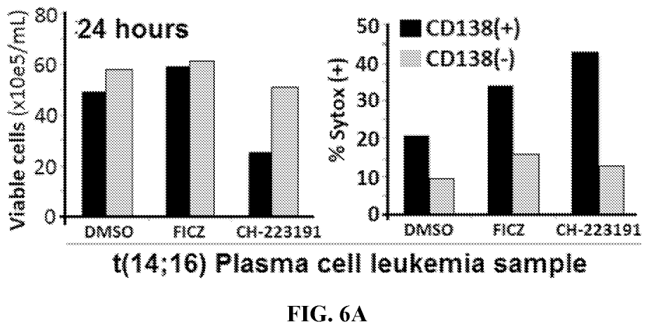

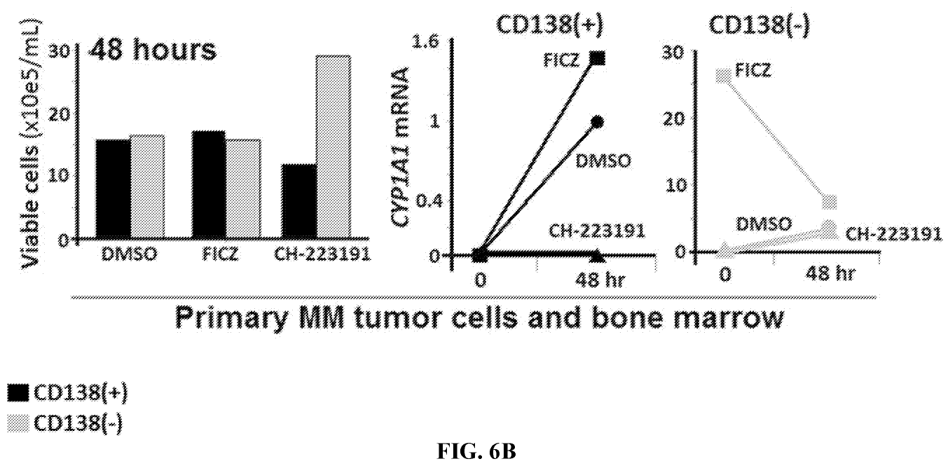

AHR antagonism induces W cell loss of viability and alters MM phenotype. AHR antagonism leads to cell line death in multiple MM cell lines. In FIG. 5 (right panel). MM cells exposed to AHR antagonism showed no recovery in proliferation after washout. Of note, the MM1R cell line, which is resistant to dexamethasone-mediated lysis, still shows sensitivity to AHR antagonism similar to the dexamethasone-sensitive MM1S line. Similarly, in primary MM samples obtained from patients, AHR antagonism led to death of CD138(+) MM tumor cells while sparing autologous CD138(−) cells. Representative results from 2 patients indicate efficacy even in samples from high risk MM cases (FIG. 6, left panel), and that transcriptional activity of AHR is suppressed by AHR antagonism in primary MM cells (right panels). AHR antagonism does not lead to complete loss of viability in all treated MM cells; however, interestingly, these surviving MM cells showed changes in morphology and immunophenotype. MM cells treated with an AHR antagonist showed reduction in size and increased expression of CD38 (1.3-2.1 fold), CD56 (1.7-12.6 fold), and CD138 (1.6-3.8 fold).

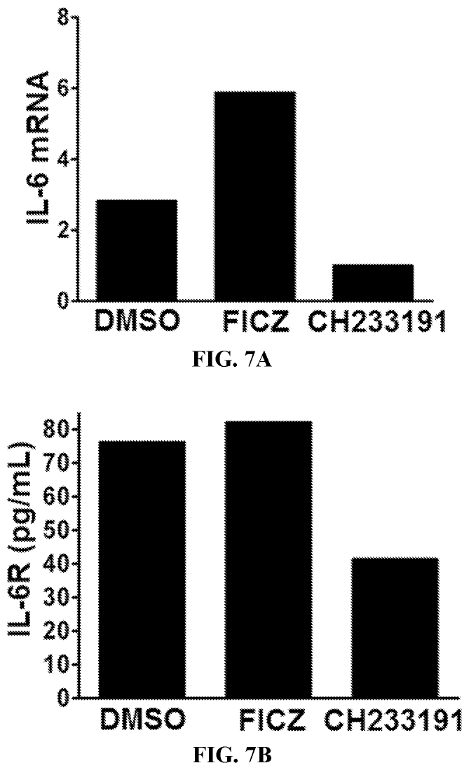

AHR antagonism suppresses MM cytokine signaling. IL-6, perhaps more than any other cytokine, has been strongly implicated in MM pathobiology. AHR antagonism leads to a reduction in IL-6 transcription in MM cells as well as a reduction in soluble IL-6 receptor produced by MM cells (FIG. 7).

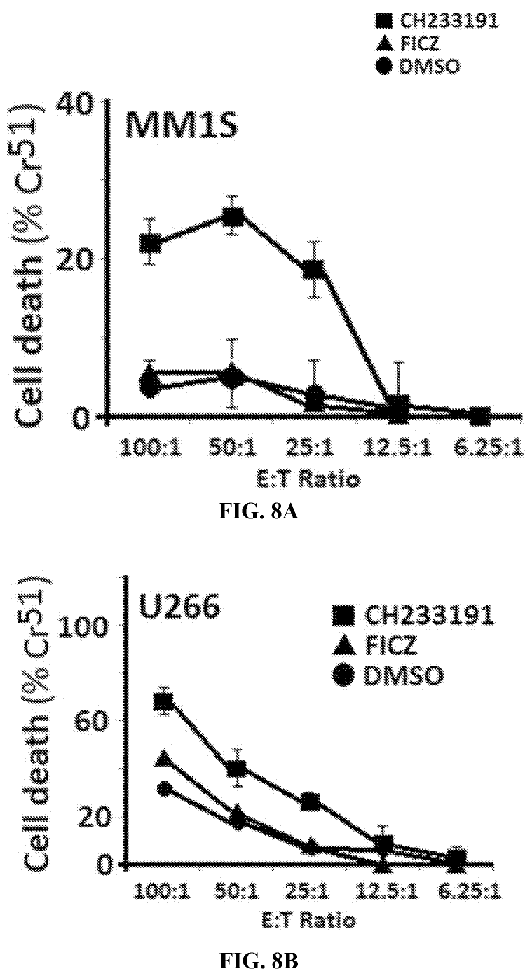

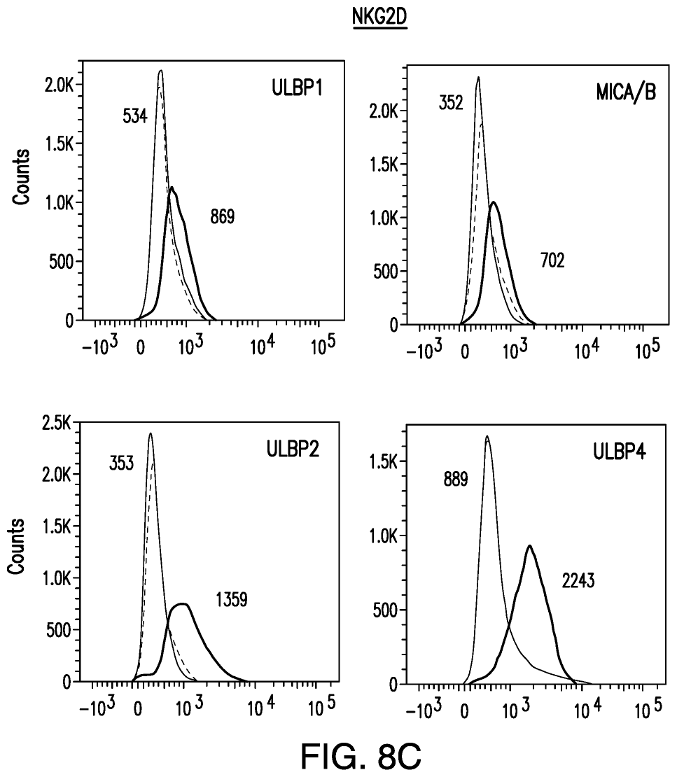

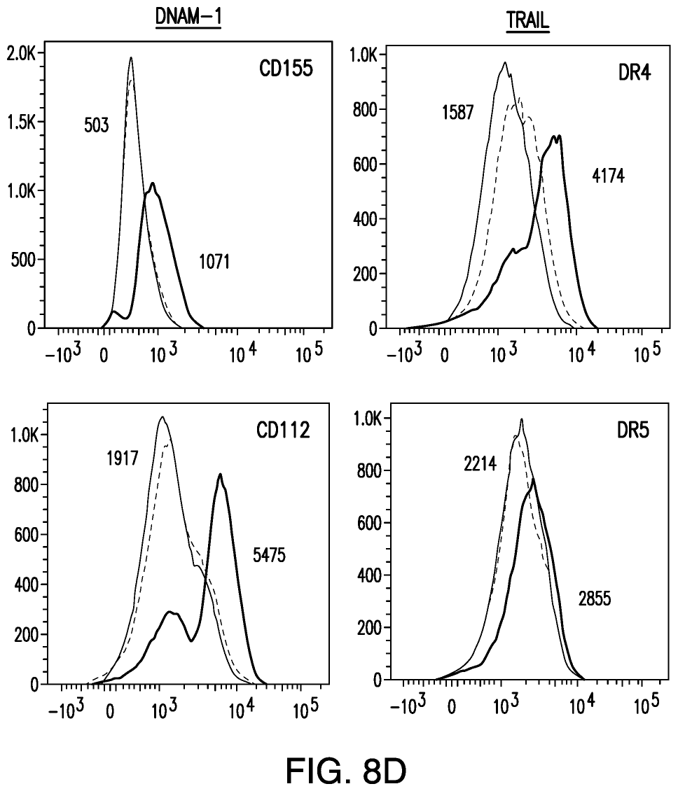

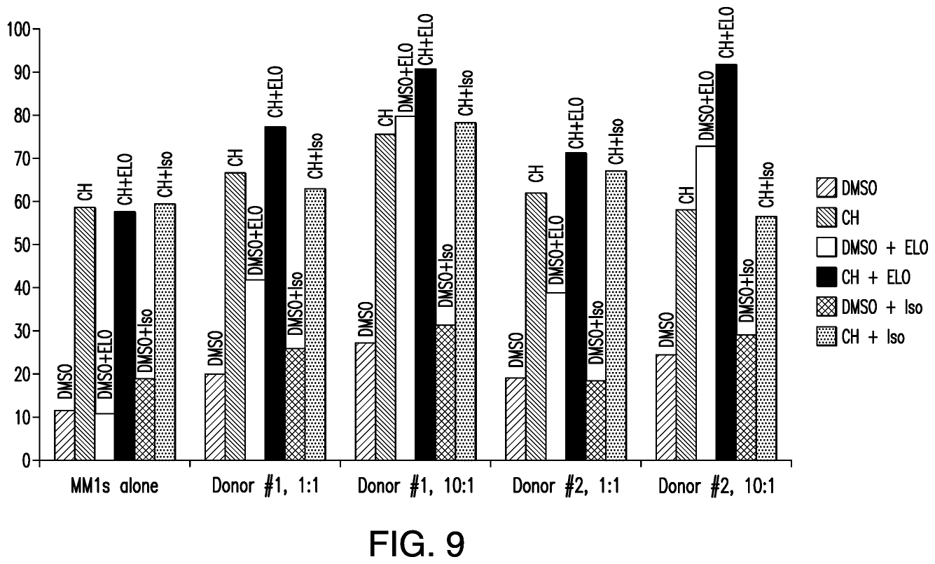

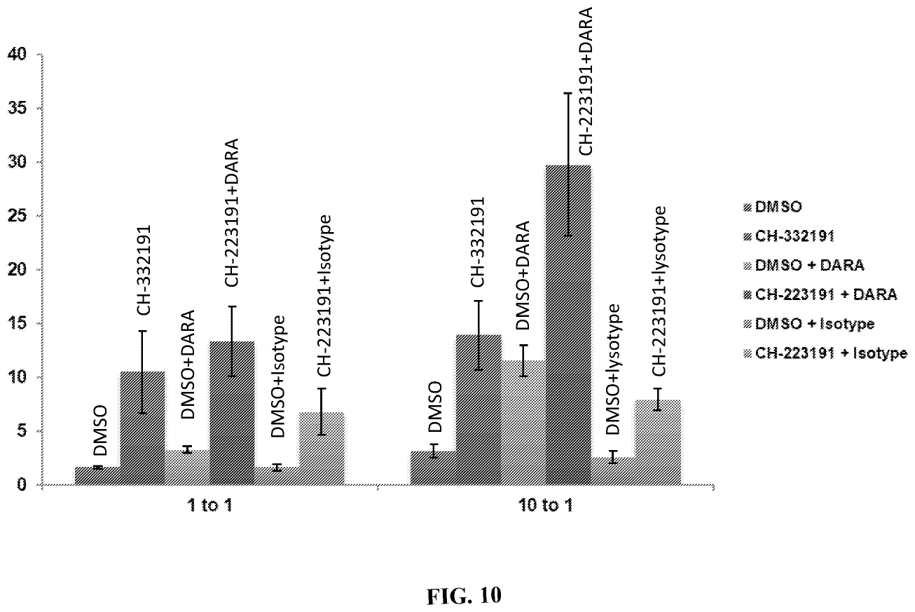

AHR antagonism sensitizes MM cells to NK-cell mediated lysis. MM cells that remain after pre-exposure to an AHR antagonist (5 days) show increased susceptibility to NK-cell cytotoxicity. As shown in FIG. 8 (top panels), this effect appears to correlate with AHR expression in MM cells. The MM1S cell line expresses relatively high AHR levels and shows significant direct sensitivity (loss of viability) to AHR antagonism but is also relatively resistant to NK cell-mediated lysis. The U266 cell line expresses relatively less AHR than other MM cell lines, and while AHR antagonism induces less direct lysis, there appears to be a greater absolute effect on susceptibility to NK-cell mediated lysis. As shown in FIG. 8 (lower panels), AHR antagonism enhances MM tumor cell expression of a number of ligands for the activating NK cell receptors NKG2D, DNAM-1, and TRAIL.

AHR antagonism enhances MM expression of antigens targetable by therapeutic monoclonal antibodies. In addition to the above immunophenotypic alterations induced by AHR antagonism, the expression of a number of proteins against which therapeutic monoclonal antibodies are available is also increased. These are summarized in Table 1 below and, combined with the data herein, suggest that AHR antagonism can further enhance the efficacy of tumor antigen-directed, therapeutic monoclonal antibody-based therapy as an additional means of potential anti-myeloma efficacy. Accordingly, NK-cell mediated killing of MM tumor cells is tested by combining AHR antagonism of effector cells and MM targets with therapeutic monoclonal antibodies.

TABLE 1
AHR inhibition enhances surface expression of MM antigens
targetable by therapeutic monoclonal antibodies either
already approved or in development currently.

| Antigen | MFI (DMSO) | MFI (CH233191) | Antibody |
|---|---|---|---|
| SLAMF7 (CS1) | 1946 | 2999 | Elotuzumab |
| CD38 | 2959 | 6110 | Daratumumab, |
| | | | Isatuximab |
| CD20 | 321 | 753 | Rituximab |
| CD74 | 761 | 1367 | Milatuzumab |

A novel liposomal formulation of CH233191. An experiment of CH233191 administered by oral gavage in a disseminated murine MM model led to an apparent reduction in MM tumor cell burden in 2 of 5 mice versus vehicle-treated controls, with no obvious toxicities. However, despite this effect, all mice died of progressive MM. The oral bioavailability of CH233191 was questionable in this system, given difficulties with solubilization of the agent in corn oil for delivery by gavage. Liposomes are phospholipid bilayer vesicles utilized as therapeutic drug carriers to improve efficacy/tolerability. This approach is believed to be especially helpful to solubilize hydrophobic agents (such as CH233191), extend circulation half life, and target tumors based on enhanced permeability and retention. Members of our group have established expertise in this area. (Zhou Eur J Pharm Sci 2014). A specific liposomal formulation composed of DOPC/Chol/m-PEG-DSPE prepared by co-dissolution of the drug with lipid excipients in ethanol has been tested, followed by rapid dilution and polycarbonate membrane extrusion to regulate particle size. Liposomes were then purified and concentrated by tangential flow diafiltration. Particle size, determined by dynamic light scattering, indicates that liposomal CH233191 is approximate 125 nm in size and stable for at least one week at 4° centigrade. The formulation has a 1:20 ratio of CH233191 to lipid and achieved a drug concentration in liposome of 1-2 mg/mL.

Characterization of the in vitro effects of AHR antagonism on MM cells and immune cells. AHR antagonism can directly affect MM cell viability and, in parallel, potentially facilitate development and function of normal immune cells. AHR expression and function can be investigated across the stages of MM, the mechanisms by which AHR antagonism induces MM cell death and phenotype elucidated, and the possible processes by which AHR antagonism enhances immune-mediated anti-MM effects characterized.

MM is known to proceed from a precursor MGUS stage to an asymptomatic form (Smoldering Multiple Myeloma, “SMM”) to clinically evident MM characterized by end-organ damage with hallmark features (hypercalcemia, renal dysfunction, anemia, and lytic bone disease). Malignant plasma cells from patient bone marrow aspirates are enriched using a positive, magnetic selection technique and AHR expression is assayed by quantitative reverse-transcription (RT) PCR and Western blotting. Treatment of samples with AHR agonist (FICZ) and antagonist (CH233191) are utilized to determine the functional status of AHR in these samples with measurement of CYP1A1 levels as an established biomarker of function. Effects on cell viability are studied by microscopy (trypan blue exclusion), MTS assay, and Sytox staining by flow cytometry. In parallel, ELISAs for KYN and KYNA are conducted to determine whether malignant plasma cells vary in their production of endogenous ligands as a function of disease state (MGUS, SMM, MM).

Whole bone marrow aspirates from patients with active MM is collected and treated in control conditions or with AHR agonist (FICZ) or antagonist (CH233191). The MM tumor cell population is defined for each patient (percent involvement) of marrow and immunophenotype at baseline. Formal dose and exposure experiments are conducted to characterize tumor cell viability and means of MM cell death. A systematic approach in primary samples identifies an optimal dose and exposure interval for maximal tumor cell death. The effects of AHR agonism and antagonism on MM cell viability as a function of cytogenetic/FISH mutations present is assessed in each sample and an attempt to identify mutations which may confer especially susceptible and/or resistant MM subtypes to AHR antagonism in the tumor microenvironment carried out. Experiments include studies to evaluate apoptosis, necrosis and autophagy as possible mechanisms of loss of viability in response to AHR antagonism. Enriched samples are used to study apoptosis pathways by Western blotting in addition to flow cytometric-based assays.

AHR antagonism can alter the production, secretion and sensitivity to ambient cytokines in the MM microenvironment. In MM cell lines and primary MM cells, the effects of AHR agonism and antagonism are studied by ELISA and cytokine bead array on cytokines critical to MM homeostasis and under the control of AHR (IL-1β, IL-21, TGF-β and IL-6). In whole marrow aspirates and peripheral blood samples from patients with MM, the effects of AHR agonism and antagonism on these cytokine levels are compared as well. In addition, the levels of Th1, Th2, and Th17 cytokines are studied by bead array in patient samples and compare these to peripheral blood samples from healthy donors.

AHR expression has been characterized in a number of MM cell lines known also to express c-myc (Drexler Leuk Res 2000), c-maf (Rasmussen Leuk Lymphoma 2003), and NF-KB (Tai Cancer Res 2006). Treating with AHR agonist or antagonist, AHR mediation of expression of these factors can be assessed by transcript and protein using CYP1A1 as a validated functional reporter in MM. The relationships between AHR and c-myc can be systematically characterized using (10058-F4 (Huang Esp Hematol 2006; Sigma Aldrich), c-maf (Nivalenol, Santa Cruz Biotechnology), and NF-KB RelA (Takata J Biol Chem 2004; Novus Biologicals) and RelB (SN52 Xu Mol Cancer Ther 2008) subunits using respective small molecule inhibitors and siRNA silencing strategies. These results shed light on rational combinations of AHR antagonism with established clinical treatment strategies.

The expression of AHR can be systematically studied by intracellular flow cytometry, real time PCR, and Western blot in B, T, and NK cell subsets in peripheral blood and bone marrow aspirates from patients with MM. AHR expression is characterized in lymphocytes from patients with MGUS, smoldering MM and active MM. Serial samples from patients are studied, whilst in the state of active disease and from samples taken in clinical remission to determine whether changes in AHR are observed as a function of disease activity. The functional activity of AHR in these lymphocyte subsets is studied by evaluating the cytoplasmic and nuclear fractions present as compared to in the normal setting as well as the functional status using CYP1A1 expression in normal lymphocyte subsets.

Data shows that AHR antagonism can augment NK cell maturation and function and increases the susceptibility of MM cells to immune-mediated lysis. The increased expression of targetable antigens for ADCC also shows a strategy to enhance the efficacy of this specific therapeutic approach in MM, as well. A series of in vitro studies are conducted in which immune effector cells and/or MM cell targets are pre-treated with vehicle or CH233191 prior to cytotoxicity co-culture assay. Accounting for the baseline, direct effect of AHR antagonism on MM cell viability, any additive rate of target cell death in the combinatorial condition can be studied, in particular when both effectors and targets are pre-exposed to CH233191. Using validated techniques (Benson Blood 2010), the effects of CH233191 on immune effector cell expansion, expression of activating receptors, and abilities to traffic to, acquire, and lyse MM targets is studied. These experiments are conducted in the presence of therapeutic monoclonal antibodies in order to assess whether AHR antagonism may enhance ADCC-mediated immunotherapy (e.g., Elotuzumab, Daratumumab/Isatuximab/Rituximab/Milatuzumab).

### Example 2: Anti-MM Effects of Liposomal CH233191

The MTD of intraperitoneal (IP) liposomal CH233101 in n=25 ICD mice is evaluated. Then the IP MTD dose is tested in intravenous (IV) administration. Injection volumes can range from 50-200 uL for IP administration and 50-100uL for IV administration. The dosing solution concentration can be 2 mg/mL. Endpoint signs in the animals are recorded, and they are observed for acute toxicities in the first 24 hours post-dosing.

Optimization of a strategy to measure free and liposomal CH233191 in mouse plasma can be used in interpretation of results in MM disease models. An analytic assay is developed and validated. Results are cross validated in MM cells and bone marrow by LC-MS/MS assay to assess linearity, accuracy, precision, and stability for both free and liposomal CH233191 in plasma, tumor and marrow.

An in vivo model has been developed in NSG mice which recapitulates many sequelae of the disease, including disseminated MM bone marrow involvement and end-organ manifestations of MM (Chu Leukemia 2014). The IP and IV MTDs are tested in NSG mice to verify findings in the ICR mice.

This set of experiments guides decisions on optimizing dosing, delivery and administration. Utilizing the derived MTD, mice are treated and plasma collected, tumor samples, and bone marrow from all animals for analysis via LC-MS/MS. The PK (T_{1/2}, clearance, AUC) and PD of liposomal CH233191 are characterized in comparison with free CH233191 given orally. The single dose PK study helps to guide modeling of in vivo achievable drug concentrations by delivery route as well as Cₘₐₓ, T_{1/2}, area of distribution, and other critical PK parameters. These data guide evaluations for PK and PD endpoints.

Guided by the findings of the above work, formal testing for efficacy is done in the murine model of MM. Details of the murine MM model have been published (Benson Blood 2010). In brief, this model effectively recapitulates human disease as shown by dissemination in the bone marrow, lytic bone disease, production of serum paraprotein, and death. MM cells are identifiable via bioluminescent imaging as well as by flow cytometry through expression of GFP protein. 10 mice receive liposomal CH233191 and 10 receive control (empty liposome formulation). Mice are followed for evidence of response by bioluminescence imaging and serial measurement of serum MM protein. After 30 days, mice are sacrificed and evaluation of CYP1A1 levels in MM cells is compared between control and treated mice as a validated PD marker to confirm CH233191 functional effects in MM cells. AHR antagonism via liposomal CH233191 administered under guidance from experiments can lead to an in vivo anti-MM tumor effect.

### Example 3: Preparation of Liposomal CH223191

Liposomal CH233191was prepared by thin film hydration and extrusion method. The lipid composition used was DOPC/CHOL/mPEG-DSPE/Tween-80 at molar ratio of 80/15/3/2. A stock solution (25 mg/ml) of CH233191 was prepared by dissolving it in DMSO. The CH233191 to lipids ratio used was 1/20. Briefly, lipids were dissolved in ethanol and mixed with CH233191 from the stock solution and dried into a thin film with rotary evaporation in a round bottom flask at 40° C. under vacuum. The lipid film was then hydrated with phosphate-buffered saline (PBS, pH 7.4). The lipid suspension was extruded three times each through 0.2 and then 0.1 μm pore size polycarbonate membranes on a nitrogen-driven Lipex lipid extruder (Northern Lipids Inc.). The lipid suspension was then purified on a Sepharose CL-4B column to remove any unencapsulated CH233191. Then, 10% sucrose was added into the solution, which is then stored frozen.

The particle size of liposomal CH233191 was determined by dynamic light scattering on a model 370 Nicomp Submicron Particle Sizer (NICOMP, Santa Barbara, Calif.). CH233191 encapsulation efficiency was determined by UV spectrometry at the wavelength of 360 nm.

The results were that the particle size of liposomal CH233191 was 125.7±5.3 nm, and the encapsulation efficiency of CH233191 in liposomes was 90.3±3.9%.

### REFERENCES

1. Denison M S, Pandini A, Nagy S R, Baldwin E P, Bonati L. Ligand binding and activation fo the Ah receptor. Chem Biol Interact 2002; 141(1-2):3-24.

2. Juliard W, Fechner J H, Mezrich J D. The aryl hydrocarbon receptor meets immunity: friend or foe? A little of both. Front Immunol 2014; 5:1-6.

3. Sherr D H, Monti S. The role of the aryl hydrocarbon receptor in normal and malignant B cell development. Semin Immunopathol 2013; 35:705-16.

4. Quintana F J, Basso A S, Iglesias A H, Korn T, Farez M F, Bettelli E, et al., Control of T(reg) and T(H)17 cell differentiation by the aryl hydrocarbon receptor. Nature 2008; 453(7191): 65-71.

5. Hughes T L, Briercheck E L, Freud A G, Trotta R, McClory S, Scoville S D, Keller K, Deng Y, Cole J, Harrison N, Mao C, Zhang J, Benson D M, Yu J, Caligiuri M A. The transcription factor AHR prevents the differentiation of stage 3 innate lymphoid cell subset to natural killer cells. Cell Reports 2014; 8(1): 150-62.

6. Opitz C A, Litzenburger U M, Sahm F, Ott M, Tritschler I, Trump S, et al., An endogenous tumour-promoting ligand of the aryl hydrocarbon receptor. Nature 2011; 478(7368): 197-203.

7. Richmond O, Ghotbaddini M, Allen C, Walker A, Zahir S, Powell J B. The aryl hydrocarbon receptor is constitutively active in advanced prostate cancer cells. PLoS One 2014; 9(4): e95058.

8. Safe S, Lee S O, Jin U H. Role of the aryl hydrocarbon receptor in carcinogenesis and potential as a drug target. Toxicol Sci 2013; 135(1): 1-16.

9. Fan Y, Boivin G P, Knudsen Es, Bebert D W, Xia Y, Puga A. The aryl hydrocarbon receptor functions as a tumor suppressor in liver carcinogenesis. Cancer Res 2010; 70(1): 212-20.

10. Ikuta T, Kobayashi Y, Kitazawa M, Shiizaki K, Itano N, Noda T, Pettersson S, Poellinger L, Fujii-Kuriyama Y, Taniguchi S, Kawajiri K. ASC-associated inflammation promotes cecal tumorigenesis in aryl hydrocarbon receptor-deficient mice. Carcinogenesis 2013; 34(7): 1620-7.

11. Murray I A, Patterson A D, Perdew G H. Aryl hydrocarbon receptor ligands in cancer: friend and foe. Nat Rev Cancer 2014; 14; 801-14.

12. Quintana P J, Delfino R J, Korrick S, Ziogas A, Kutz F W, Jones E L, Laden F, Garshick E. Adipose tissue levels of organo-chlorine pesticides nad polychlorinated bipheyls and the risk of non-Hodgkin lymphoma. Envion Heal Perspect 2002; 112(8): 854-61.

13. Hardel L, Eriksson M, Nordstrom M. Exposure to pesticides as a risk factor for non-Hodgkin's lymphoma and hairy cell leukemia: pooled analysis of two Swedish case-control studies. Leuk Lymphoma 2002; 43(5): 1043-9.

14. Viel J F, Arveux P, Baverel J, Cahn J Y. Soft-tissue sarcoma and non-Hodgkin's lymphoma clusters around a municipal solid waste incinerator with high dioxin emission levels. Am J Epidemiol 2000; 152(1): 13-9.

15. Consonni D, Pesatori A C, Zocchetti C, Sindaco R, D'Oro Lc, Rubagotti M, Bertazzi P A. Mortality in a population exposed to dioxin after the Seveso, Italy accident in 1976: 25 years of follow up. Am J Epidemiol 2008; 167(7): 847-58.

16. Available online at http://books.nap.edu/openbook.php?record_id=13166

17. DiNatale B C, Schroeder J C, Francey L J, Kusnadi A, Perdew G H. Mechanistic insights into the events that lead to synergistic induction of interleukin 6 transcription upon activation of the aryl hydrocarbon receptor and inflammatory signaling. J Bio Chem 2010; 285:24388-97.

18. Lahoti T S, Hughes J M, Kusnadi A, John K, Zhu B, Murray I A, Gowda K, Peters J M, Amin S G, Perdew G H. Aryl hydrocarbon receptor antagonism attenuates growth factor expression, proliferation and migration in fibroblast-like synoviocytes from patients with rheumatoid arthritis. J Pharmacol Exp Ther 2014; 348(2): 236-45.

19. Gramatzki D, Pantazis G, Schittenhelm J, Tabatabai G, Kohle C, Wick W, Schwarz M, Weller M, Tritschler I. Aryl hydrocarbon receptor inhibition downregulates TGF-□/SMAD pathway in human glioblastoma cells. Oncogene 2009; 28:2593-605.

20. Apetoh L, Quintana F J, Pot C, Joller N, Xaio S, Kumar D, Burns E J, Sherr D H, Weiner H L, Kuchroo V K. The aryl hydrocarbon receptor interacts with c-Maf to promote the differentiation of type 1 regulatory T cells induced by IL-27. Nat Immunol 2010; 11(9): 854-61.

21. Hollingshead B D, Beischlag, T V, Dinatale B C, Ramadoss P, Perdew G H. Inflammatory signaling and aryl hydrocarbon receptor mediate synergistic induction of interleukin 6 in MCF-7 cells. Cancer Res 2008; 68: 3609-17.

22. Hurt E M, Wiestner A, Rosenwald A, Shaffer A L, Campo E, Grogan T, Bergsagel P L, Kuehl W M, Staudt L M. Overexpression of c-maf is a frequent oncogenic event in multiple myeloma that promotes proliferation and pathological interactions with bone marrow stroma. Cancer Cell 2004; 5(2): 191-9.

23. Monteiro P, Gilot D, Le Ferrec E, Lecureur V, N'diaye M, Le Vee M, Podechard N, Pouponnot C, Fardel O. AhR- and c-maf-dependent induction of beta7-integrin expression in human macrophages in response to environmental polycyclic aromatic hydrocarbons. Biochem Biophys Res Commun 2007; 358(2): 442-8.

24. Chesi M, Bergsagel P L. Molecular pathogenesis of multiple myeloma: basic and clinical updates. Int J Hematol 2013; 97(3): 313-23.

25. Vogel C F, Khan E M, Leung P S, Gershwin M E, Chang W L, Wu D, Haarmann-Stemmann T, Hoffmann A, Denison Ms. Cross-talk between aryl hydrocarbon receptor and the inflammatory response: a role for nuclear factor-KB. J Biol Chem 2014; 289(3): 1866-75.

26. Tian Y, Ke S, Denison M S, Rabson A B, Gallo M A. Ah receptor and NF-kappaB interactions, a potential for dioxin toxicity. J Biol Chem 1999; 274(1): 510-5.

27. Cormier F, Monjanel H, Fabre C, Bilot K, Sapharikas E, Chereau F, Bordereaux D, Molina T J, AVet-Loiseau H, Baud V. frequent engagement of RelB activation is critical for cell survival in multiple myeloma. PLoS One 2013; 8(3): e59127.

28. Vogel C F, Li W, Wu D, Miller J K, Sweeney C, Lazennec G, Fujisawa Y, Matsumura F. Interaction of aryl hydrocarbon receptor and NK-KB subunit RelB in breast cancer is associated with interleukin-8 overexpression. Arch Biochem Biophys 2011; 512(1): 78-86.

29. Vogel C F, Sciullo E, Matsumura F. Involvement of RelB in aryl hydrocarbon receptor-mediated induction of chemokines. Biochem Biophys Res Commun 2007; 363(3): 722-6.

30. Kim D W, Gazourian L, Quadri S A, Romieu-Mourez R, Sherr D H, Sonenshein G E. The RelA NF-kappaB subunit and the aryl hydrocarbon receptor (AhR) cooperate to transactiate the c-myc promoter in mammary cells. Oncogene 2000; 19(48): 5498-506.

31. Gilitza I C, Lu G, Shah R, Bashir Q, Shah N, Champlin R E, Shah J, Orlowski R Z, Qazilbash M H. Chromosome 8q24.1/c-MYC abnormality: a marker for high-risk myeloma. Leuk Lymphoma 2014; Aug. 18: 1-6—e-pub ahead of print.

32. Pichiorri F, Suh S S, Rocci A, De Luca L, Taccioli C, Santhanam R, Zhou W, Benson D M Jr, Hofmeister C C, Alder H, Garofalo M, Di Leva G, Volinia S, Lin H J, Perrotti D, Kuehl M, Ageilan R I, Palumbo A, Croce C M. Downregulation of p-53-inducible microRNAs 192, 194, and 215 impairs the p53/MDM2 autoregulatory loop in multiple myeloma development. Cancer Cell 2010; 18(4): 367-81.

33. Gordon M W, Yan F, Zhong X, Mazumder P B, Xu-Monette Z Y, Zou D, Young K H, Ramos K S, Li Y. Regulation of p-53 targeting microRNAs by polycyclic aromatic hydrocarbons: implications in the etiology of multiple myeloma. Mol Carcinog 2014; May 6—e pub ahead of print.

34. Mariani E, Fontana F, Mari S, Manteiga J, Marcatti M, Napoli N, Camnasio F, Fraschini G, Caneva E, Sitia R, Musco G, Ciceri F, Cenci S. Metabolics identifies plasma biomarkers of multiple myeloma and progression. Bone Abstracts 2013; 1 PP 146a.

35. Bonanno G, Mariotti A, Folgiero V, Natale D, De Rosa L, Majolino I, Novarese L, Rocci A, Gambella M, Scambia G, Palumbo A, Locatelli F, De Cristofaro R, Rutella S. Indoleamine 2,3-dioxygenase 1 (IDO1) activity correlates with immune system abnormalities in multiple myeloma. J Transl Med 2012; Dec. 11; 10:247.

36. Zdzisinska B, Wejksza K, Walter-Croneck A, Turski W A, Kandefer-Szerszen M. Kynurenic acid in blood and blood marrow plasma of monoclonal gammopathy of undetermined significance (MGUS) and multiple myeloma (MM) patients. Leuk Res 2010; 34: 38-45.

37. Rutella S, Bonanno G, Corallo M, Mariotti A, Procoli A, Majolino I, DeCristofaro R. Expression of indoleamine 2,3-dioxygenase (IDO) in multiple myeloma promotes the generation of regulatory T cells: Investigations into the role of hepatocyte growth factor. J Immunol 2009; 182 (meeting abstract supplement) 40.1.

38. Hughes T L, Ciarlariello D, Bakan C E, Benson D M Jr. The role of aryl hydrocarbon receptor activation in multiple myeloma. Clin Lymphoma Myeloma 2013; 13(1): S61-2.

39. Boitano A E, Wang J, Romeo R, Bouchez L C, Parker A E, Sutton S E, Walker J R, Flaveny C A, Perdew G H, Denison M S, Schultz P G, Cooke M P. Aryl hydrocarbon antagonists promote the expansion of human hematopoietic stem cells. Science 2010; 329: 1345-8.

40. Negishi T, Kato Y, Ooneda O, Mimura J, Takada T, Mochizuki H, Yamamoto M, Fujii-Kuriyama Y, Furusako S. Effects of aryl hydrocarbon receptor signaling on the modulation of Th1/Th2 balance. J Immunol 2005; 175(11): 7348-56.

41. Hughes T, Becknell B, Freud A G, McClory S, Briercheck E, Yu J, Mao C, Giovenzana C, Nuovo G, Wei L, Zhang X, Gavrilin Ma. Wewers M D. Caligiuri M A. Interleukin-1beta selectively expands and sustains interleukin-22+ immature human natural killer cells in secondary lymphoid tissue. Immunity 2010; 32(6): 803-14.

42. Roeven M W H, Cany J, Maas F, Kohela A, Joop J, Blijlevens N M A, Schaap N P M, Dolstra H. the aryl hydrocarbon receptor antagonist Stemreginin 1 stimulates expression of NK cell related transcription factors, thereby it facilitates generation of highly functional NK cells in vitro. Blood 2014; 124 (21, Annual Meeting Abstracts): 3383a.

43. Shapiro-Shelef, M, Calame K. Plasma cell differentiation and multiple myeloma. Curr Opin Immunol 2004; 16(2): 226-34.

44. Zhan F, Tian E, Bumm K, Smith R, Barlogie B, Shaughnessy J. Gene expression profiling of human plasma cell differentiation and classification of multiple myeloma based on similarities to distinct stages of late-stage B-cell development. Blood 2003; 101(3): 1128-40.

45. Boise L H, Kaufman J L, Bahlis Nj, Lonial S, Lee K P. The Tao of myeloma. Blood 2014; 124(12): 1873-9.

46. Kawano Y, Fukjiwara S, Wada N, Izaki M, Yuki H, Okuno Y, et al., Multiple myeloma cells expressing low levels of CD138 have an immature phenotype and reduced sensitivity to lenalidomide. Int J Oncol 2012; 41(3): 876-84.

47. Matsui W, Huff C A, Wang Q, Malehorn M T, Barber J, Tanhehco Y, et al., Characterization of clonogenic multiple myeloma cells. Blood 2004; 103(6): 2332-6.

48. Pellat-Deceunynck C, Bataille R. Normal and malignant plasma cells: proliferation, differentiation, and expansions in relation to CD45 expression. Blood Cells Mol Dis 2004; 32(2): 293-301.

49. Medina F, Sugundo C, Campos-Caro A, Gonzalez-Garcia I, Brieva J A. The heterogeneity shown by human plasma cells from tonsil, blood and bone marrow reveals graded stages of increasing maturity but local profiles of adhesion molecule expression. Blood 2002; 99(6): 2154-61.

50. Matwui W, Wang Q, Barber J P, Brennan S, Smith B D, Borrello I, et al., Clonogenic multiple myeloma progenitors, stem cell properties and drug resistance. Cancer Res 2008; 68(1): 190-7.

51. Drexler H G, Matsuo Y. Malignant hematopoietic cell lines: in vitro models for the study of multiple myeloma and plasma cell leukemia. Leuk Res 2000; 24(8): 681-703.

52. Rasmussen T, Knudsen L M, Dahl I M S, Johnsen H E. C-MAF oncogene dysregulation in multiple myeloma: frequency and biological relevance. Leuk Lymphoma 20003; 40(10): 1761-6.

53. Tai Y T, Li X F, Breitkreutz I, Song W, Neri P, Catley L, et al., Role of B-cell activating factor in adhesion and growth of human multiple myeloma cells in the bone marrow microenvironment. Cancer Res 2006; 66(13): 6675-82.

54. Benson D M, Bakan C E, Mishra A, Hofmeister C C, Efebera Y, Becknell B, et. al., The PD-1/PD-L1 axis modulates the natural killer cell versus multiple myeloma effect: a therapeutic target for CT-011, a novel monoclonal anti-PD-1 antibody. Blood 2010; 116:2286-94.

55. Chu J, Deng Y, Benson D M, He S, Hughes T, Zhang J, et al., CS1-specific chimeric antigen receptor (CAR)-engineered natural killer cells enhance in vitro and in vivo antitumor activity against human multiple myeloma. Leukemia 2014; 28:917-27.

56. Rajikumar et al. International Myeloma Working Group updated criteria for the diagnosis of multiple myeloma. Lancet Oncol. 2014 November; 15(12):e538-48.

57. Parks et al. In Silico Identification of an Aryl Hydrocarbon Receptor Antagonist with Biological Activity In Vitro and In Vivo. Mol Pharm 2014; vol 86: p 593-608.

58. Kadow S, Jux B, Zahner S P, Wingerath B, Chmill S, Clausen B E, Hengstler J, Esser C. Aryl hydrocarbon receptor is critical for homeostatis of invariant gamma delta T cells in the murine epidermis. J Immunol 2011; 187(6): 3104-10.

59. Veldhoen M, Hirota K, Westendorf A M, Buer J, Dumoutier L, Renauld J C, Stockinger B. The aryl hydrocarbon receptor links TH17-cell-mediated autoimmunity to environmental toxins. Nature 2008; 453(7191): 106-9.

60. Prabhala R H, Pelluru D, Fulciniti M, Prabhala H K, Nanjappa P, Song W, Pai C, Amin S, Tai Y T, Richardson P G, Ghobrial I M, Treon S P, Daley J F, Anderson K C, Kutok J L, Munshi N C. Elevated IL-17 produced by TH17 cells promotes myeloma cell growth and inhibits immune function in multiple myeloma. Blood 2010; 115(26): 5385-92.

61. Fang Z Z, Krausz K W, Nagaoka K, Tanaka N, Gowda K, Amin S G, Perdew G H, Gonzalez F J. In vivo effects of the pure aryl hydrocarbon receptor antagonist GNF-351 after oral administration are limited to the gastrointestinal tract. Brit J Pharmacol 2014; 171(7): 1735-46

1. A composition comprising CH233191 and a liposome.

2. The composition of claim 1, wherein said composition is in an injectable form.

3. The composition of claim 1, wherein said composition is in an intravenous form.

4. The composition of claim 1, wherein said composition is in a format for intraperitoneal injection.

5. The composition of claim 1, wherein said composition allows for controlled release of CH233191.

6. The composition of claim 1, wherein the composition further comprises one or more additional therapeutic agents.

7. The composition of claim 6, wherein said additional therapeutic agent comprises daratumumab, elotuzumab, silotuximab, isatuximab, rituximab, or milatuzumab.

8. The composition of claim 1, wherein the composition is used to treat multiple myeloma.

9. The composition of claim 1, wherein the particle size of liposomal CH233191 is greater than 100 nm.

10. The composition of claim 1, wherein the encapsulation efficiency of CH233191 in liposomes is greater than 90%.

11. A method of treating a plasma cell neoplasm, comprising:
a. diagnosing a subject with a plasma cell neoplasm; and
b. administering to the subject a substance that down-regulates aryl hydrocarbon receptor (AHR), thereby treating a plasma cell neoplasm.

12. The method of claim 11, wherein said plasma cell neoplasm is multiple myeloma.

13. The method of claim 12, wherein the subject has been diagnosed with multiple myeloma.

14. The method of claim 13, wherein the subject has been diagnosed with multiple myeloma by diagnosis of a plasma cell tumor.

15. The method of claim 13, wherein the subject has been diagnosed with multiple myeloma by determining that at least 10% of the cells in the bone marrow are plasma cells.

16. The method of claim 11, wherein the substance that down-regulates AHR is an AHR antagonist.

17. The method of claim 16, wherein the substance that down-regulates AHR is CH233191.

18. The method of claim 17, wherein CH233191 is in a liposomal formulation.

19. The method of claim 18, wherein the substance that down-regulates AHR is stem-reginin-1.

20. The method of claim 11, further comprising:
administering to the subject one or more additional therapeutic agents for treating plasma cell neoplasms.

21-49. (canceled)
